(19) United States
(12) Patent Application Publication
(10) Pub. No.: US 2026/0014204 A1
(43) Pub. Date: Jan. 15, 2026

(54) STEM CELL-DRUG CARRIER COMPRISING MELATONIN-CONTAINING DRUG CARRIER AND STEM CELL AND USES THEREOF

(71) Applicants: PUSAN NATIONAL UNIVERSITY INDUSTRY-UNIVERSITY COOPERATION FOUNDATION, Busan (KR); Research & Business Foundation SUNGKYUNKWAN UNIVERSITY, Suwon-si (KR)

(72) Inventors: Hyung-Sik KIM, Yangsan-si (KR); Jee-Heon JEONG, Suwon-si (KR); Yoo-Jin SEO, Yangsan-si (KR); Ji-Su AHN, Yangsan-si (KR)

(21) Appl. No.: 19/220,724

(22) Filed: May 28, 2025

(30) Foreign Application Priority Data
May 28, 2024 (KR) 10-2024-0069465

(51) Int. Cl. A61K 35/28; A61K 47/69

(52) U.S. Cl. CPC A61K 35/28; A61K 47/6901

(57) ABSTRACT

The present disclosure relates to a stem cell-drug carrier including a melatonin-containing drug carrier and a stem cell, and more particularly, to a use of the stem cell for the treatment of intestinal epithelial damage disease and inflammatory bowel disease. According to the present disclosure, it was confirmed that the stem cell-drug carrier continuously released PGE2 and had excellent revival stem cell induction ability. This means that the stem cell-drug carrier of the present disclosure has an excellent intestinal epithelium regeneration effect, and the stem cell-drug carrier of the present disclosure can be used in various fields of treatment of intestinal damage disease and inflammatory bowel disease.

### CROSS-REFERENCE TO RELATED APPLICATION

This present application claims the benefit of priority to Korean Patent Application No. 10-2024-0069465, entitled “STEM CELL-DRUG CARRIER COMPRISING MELATONIN-CONTAINING DRUG CARRIER AND STEM CELL AND USES THEREOF”' filed on May 28, 2024, in the Korean Intellectual Property Office, the entire disclosure of which is incorporated herein by reference.

### FIELD

The present disclosure relates to a stem cell-drug carrier including a melatonin-containing drug carrier and a stem cell, and more particularly, to a use of the stem cell for the treatment of intestinal epithelial damage disease and inflammatory bowel disease.

### BACKGROUND

Inflammatory bowel disease (IBD) is a chronic and recurrent inflammatory disease represented by ulcerative colitis and Crohn's disease. Although the pathogenesis of inflammatory bowel disease is still unclear, genetic factors, abnormalities in the intestinal barrier function, intestinal microbiota imbalance, and environmental factors are estimated as potential causes. Since patients with inflammatory bowel disease are characterized by serious damage to the intestinal barrier, one of the main treatment targets is to promote tissue regeneration to restore the integrity of the intestinal barrier. Drugs currently used in the treatment of inflammatory bowel disease have problems such as refractoriness, resistance, and side effects, and show limitations in restoring the integrity of the intestinal mucosa and regenerating tissue.

Meanwhile, revival stem cells (revSCs) correspond to a stem cell population which has been recently newly discovered in the intestinal epithelium, and have been found to be specialized in the restoration of damaged tissue. Stem cells expressing leucine-rich repeat-containing G-protein coupled receptor 5 (Lgr5) in the crypt of the intestinal epithelium serve to replenish intestinal epithelial cells through rapid proliferation and differentiation and maintain homeostasis. However, these stem cells are easily lost by signals such as dextran sulfate sodium (DSS) and radiation exposure that cause epithelial damage. Instead, it has been found that revival stem cells expressing fetal transcripts including Clu, Ly6a (Sca-1), and Anxa1 are induced to regenerate epithelial tissue. It has been reported that the revival stem cells may be induced by prostaglandin E2 (PGE2), and PGE2 secreted from fibroblasts in the intestinal mucosa may bind to an EP4 receptor of intestinal epithelial cells to induce revival stem cells while activating YAP signaling.

### SUMMARY

Therefore, the present inventors developed ‘stem cell-drug carriers (heterospheroids)’ by mixing melatonin microspheres with 3D mesenchymal stem cells while studying treatment of intestinal epithelial damage and inflammatory bowel disease. In addition, the present inventors have confirmed that heterospheroids according to the present disclosure continuously released melatonin and PGE2 and had excellent revival stem cell induction ability, and then completed the present disclosure.

Therefore, an object of the present disclosure is to provide a stem cell-drug carrier including a melatonin-containing drug carrier; and a stem cell.

Another object of the present disclosure is to provide a method of regenerating intestinal epithelial cells, comprising: administering the stem cell-drug carrier to an individual in need thereof.

Yet another object of the present disclosure is to provide a method of inducing revival stem cells comprising: administering the stem cell-drug carrier to an individual in need thereof.

Yet another object of the present disclosure is to provide a method of treating inflammatory bowel disease, comprising: administering the stem cell-drug carrier to an individual in need thereof.

Yet another object of the present disclosure is to provide a method for preparing a cell therapeutic agent for regenerating intestinal epithelial cells, comprising: (a) preparing a melatonin-containing drug carrier by mixing and homogenizing melatonin and a polymer; and (b) preparing a stem cell-drug carrier by mixing the melatonin-containing drug carrier prepared in step (a) and stem cells.

According to the present disclosure, it was confirmed that the stem cell-drug carrier continuously released melatonin and PGE2 and had excellent revival stem cell induction ability. This means that the regenerative effect of intestinal epithelium is excellent, and thus the stem cell-drug carrier of the present disclosure can be utilized in various fields of treatment of intestinal damage disease and inflammatory bowel disease.

### BRIEF DESCRIPTION OF THE DRAWINGS

The above and other aspects, features, and advantages of the present disclosure will become apparent from the detailed description of the following aspects in conjunction with the accompanying drawings, in which:

FIG. 1A is a diagram showing a preparation process of stem cell-drug carriers (i.e., heterospheroids) according to the present disclosure.

FIG. 1B is a diagram showing results of measuring diameters of heterospheroids according to the present disclosure.

FIGS. 1C and 1D are diagrams showing results of confirming cell viability of heterospheroids according to the present disclosure through LIVE/DEAD staining.

FIG. 2A is a diagram showing results of observing intestinal epithelial organoids co-cultured with heterospheroids according to the present disclosure.

FIG. 2B is a diagram showing results of classifying intestinal epithelial organoids co-cultured with heterospheroids according to the present disclosure according to cell morphology.

FIG. 2C is a diagram showing results of analyzing the expression of intestinal epithelial markers in intestinal epithelial organoids co-cultured with heterospheroids according to the present disclosure (*: p<0.05, **: p<0.01, ***: p<0.001, ****: p<0.0001).

FIG. 2D is a diagram showing results of counting cells expressing revival stem cell markers in intestinal epithelial organoids co-cultured with heterospheroids according to the present disclosure through flow cytometry (*: p<0.05, **: p<0.01, ***: p<0.001, ****: p<0.0001).

FIG. 3A is a diagram showing results of measuring body weights in an intestinal inflammation mouse model administered with heterospheroids according to the present disclosure.

FIG. 3B is a diagram showing results of confirming survival rates in an intestinal inflammation mouse model administered with heterospheroids according to the present disclosure.

FIG. 3C is a diagram showing results of evaluating disease activity indexes in an intestinal inflammation mouse model administered with heterospheroids according to the present disclosure (*: p<0.05, **: p<0.01, ***: p<0.001, ****: p<0.0001).

FIG. 3D is a diagram showing results of measuring colon lengths in an intestinal inflammation mouse model administered with heterospheroids according to the present disclosure (*: p<0.05, ** p<0.01, ***: p<0.001, ****: p<0.0001).

FIG. 3E is a diagram showing results of H&E staining of intestinal epithelial tissue in an intestinal inflammation mouse model administered with heterospheroids according to the present disclosure.

FIG. 3F is a diagram showing results of evaluating intestinal epithelial damage in an intestinal inflammation mouse model administered with heterospheroids according to the present disclosure (*: p<0.05, **: p<0.01, ***: p<0.001, ****: p<0.0001).

FIGS. 3G to 3I are diagrams showing results of analyzing inflammatory indicators TNF-α, IL-17, and IL-10 in an intestinal inflammation mouse model administered with heterospheroids according to the present disclosure (*: p<0.05, **: p<0.01, ***: p<0.001, ****: p<0.0001).

FIG. 3J is a diagram showing results of analyzing myeloperoxidase (MPO) activity in the colon in an intestinal inflammation mouse model administered with heterospheroids according to the present disclosure (*: p<0.05, **: p<0.01, ***: p<0.001, ****: p<0.0001).

FIG. 3K is a diagram showing results of analyzing the induction of revival stem cells in an intestinal inflammation mouse model administered with heterospheroids according to the present disclosure (*: p<0.05, **: p<0.01, ***: p<0.001, ****: p<0.0001).

FIG. 4A is a diagram showing results of analyzing cells expressing a revival stem cell marker Ly6a in intestinal epithelial organoids co-cultured with a stem cell-drug carrier according to the present disclosure through flow cytometry (*: p<0.05, **: p<0.01, ***: p<0.001, ****: p<0.0001).

FIG. 4B is a diagram showing results of analyzing cells expressing a revival stem cell marker Ly6a in intestinal epithelial organoids co-cultured with a stem cell-drug carrier (quercetin) through flow cytometry.

FIG. 4C is a diagram showing results of analyzing the expression of a revival stem cell marker Cldn4 in intestinal epithelial organoids co-cultured with a stem cell-drug carrier according to the present disclosure through real-time qPCR (*: p<0.05, **: p<0.01, ***: p<0.001, ****: p<0.0001).

FIG. 4D is a diagram showing results of analyzing the expression of a revival stem cell marker Cldn4 in intestinal epithelial organoids co-cultured with a stem cell-drug carrier (quercetin) through real-time qPCR (*: p<0.05, **: p<0.01, ***: p<0.001, ****: p<0.0001).

FIG. 4E is a diagram showing results of analyzing the expression of Cldn4 in colon organoids co-cultured with a stem cell-drug carrier (quercetin) through real-time qPCR (*: p<0.05, **: p<0.01, ***: p<0.001, ****: p<0.0001).

### DETAILED DESCRIPTION

Hereinafter, the present disclosure will be described in detail.

According to an aspect of the present disclosure, the present disclosure provides a stem cell-drug carrier including a melatonin-containing drug carrier; and a stem cell.

The melatonin-containing drug carrier of the present disclosure has melatonin encapsulated in the carrier. In an embodiment of the present disclosure, it was confirmed that melatonin induced revival stem cells without an adverse effect on the cell viability of stem cells. In contrast, quercetin, which is clinically used as a therapeutic agent for inflammatory bowel disease, was proven to have an effect of treating inflammatory bowel disease, but was confirmed to have no significant effect on the induction of revival stem cells when applied together with stem cells. Accordingly, the drug carrier of the present disclosure is characterized by inducing regeneration and revival of particularly, damaged intestinal epithelial cells.

The stem cell-drug carrier of the present disclosure is characterized to be mixed with the melatonin-containing drug carrier. Due to the technical features above, the stem cell-drug carrier of the present disclosure may not only enable in vivo delivery of stem cells but also improve the release duration of the drug.

In the present disclosure, the drug carrier means a particle formed by forming a polymer coating layer based on the drug, and for convenience, may be indicated in the form of “polymer type-drug carrier”. For example, when the polymer type is poly(lactic-co-glycolic acid) (PLGA), the drug carrier is indicated as a PLGA-drug carrier.

In a specific embodiment of the present disclosure, the drug carrier may be a biodegradable polymer-drug carrier known in the art.

In a specific embodiment of the present disclosure, the drug carrier is preferably prepared with one or more polymers selected from the group consisting of polylactide-co-glycolide, polylactide-co-glycolide-co-ethylene glycol, polystyrene-co-ethylene glycol, polyethyleneimine-co-ethylene glycol, polyphosphagen-co-ethylene glycol, polylactide-co-ethylene glycol, polycaprolactone-co-ethylene glycol, polyanhydride-co-ethylene glycol, polymalic acid-co-ethylene glycol and derivatives polyalkylcyanoacrylate-co-ethylene glycol, polyhydroxybutyrate-co-ethylene glycol, polycarbonate-co-ethylene glycol and polyorthoester-co-ethylene glycol, polyethylene glycol, poly-L-lysine-co-ethylene glycol, polyglycolide-co-ethylene glycol, polymethylmethacrylate-co-ethylene glycol, polyvinylpyrrolidone-co-ethylene glycol, and copolymers thereof, and more preferably polyvinyl alcohol, but the scope of the present disclosure is not limited thereto.

The stem cell of the present disclosure is not limited thereto, but may be autologous or allogenic-derived.

In a specific embodiment of the present disclosure, the stem cell may be an embryonic stem cell, a mesenchymal stem cell or an induced pluripotent stem cell, and preferably a mesenchymal stem cell.

In the present disclosure, the embryonic stem cell (ESC) is commonly abbreviated as an ES cell, but is pluripotent and refers to a cell derived from the inner cell mass of a blastocyst, which is an early-stage embryo. For the purpose of the present disclosure, the term “ESC” is also sometimes used broadly and thus includes an embryonic germ cell.

As used herein, the term ‘mesenchymal stem cell (MSC)’ refers to a pluripotent progenitor cell before differentiation into cells of a specific organ, such as bone, cartilage, fat, tendon, nerve tissue, fibroblasts, and muscle cells.

In the present disclosure, the induced pluripotent stem cell (iPSC) is commonly abbreviated as an iPS cell, and refers to a type of normally non-pluripotent cell, such as a pluripotent stem cell artificially induced from an adult somatic cell, by inducing the “forced” expression of a specific gene.

In a preferred embodiment of the present disclosure, the mesenchymal stem cell may be derived from embryonic yolk sac, placenta, umbilical cord, umbilical cord blood, tonsil, skin, peripheral blood, bone marrow, adipose tissue, muscle, liver, nerve tissue, periosteum, fetal membrane, synovium, synovial fluid, amniotic membrane, meniscus, anterior cruciate ligament, articular chondrocytes, milk teeth, perivascular cells, trabecular bone, subpatellar fat pad, spleen or thymus, and preferably umbilical cord blood or tonsil.

The stem cells are preferably derived from humans, but may also be derived from fetuses or mammals other than humans. The mammals other than humans may be more preferably dogs, cats, monkeys, cows, sheep, pigs, horses, rats, mice, guinea pigs, or the like, and the origin is not limited.

In a specific embodiment of the present disclosure, the stem cell may be in the form of a spheroid.

In a specific embodiment of the present disclosure, the spheroid may have an average size of 50 to 300 μm, preferably an average size of 162.09 μm, but the scope of the present disclosure is not limited thereto.

In addition, the stem cell-drug carrier may include 1×10¹ to 1×10⁵ cells, preferably 1×10² to 1×10⁴ cells.

The melatonin-containing drug carrier of the present disclosure has a spherical shape in which melatonin is encapsulated in the carrier.

In addition, the stem cell-drug carrier of the present disclosure may include one or more melatonin-containing drug carriers for a single cell unit of stem cells.

In addition, it is preferable that the stem cell-drug carrier of the present disclosure is transplantable into a living body.

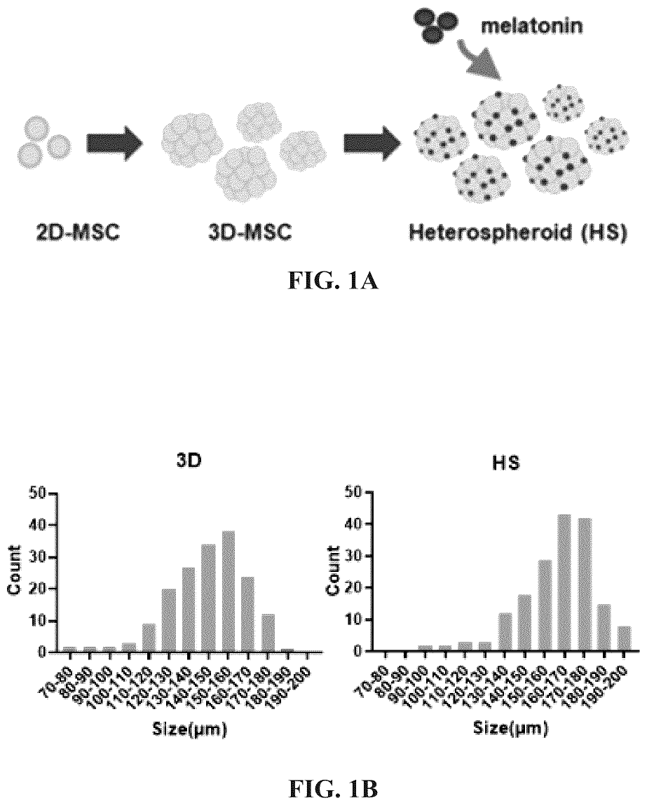

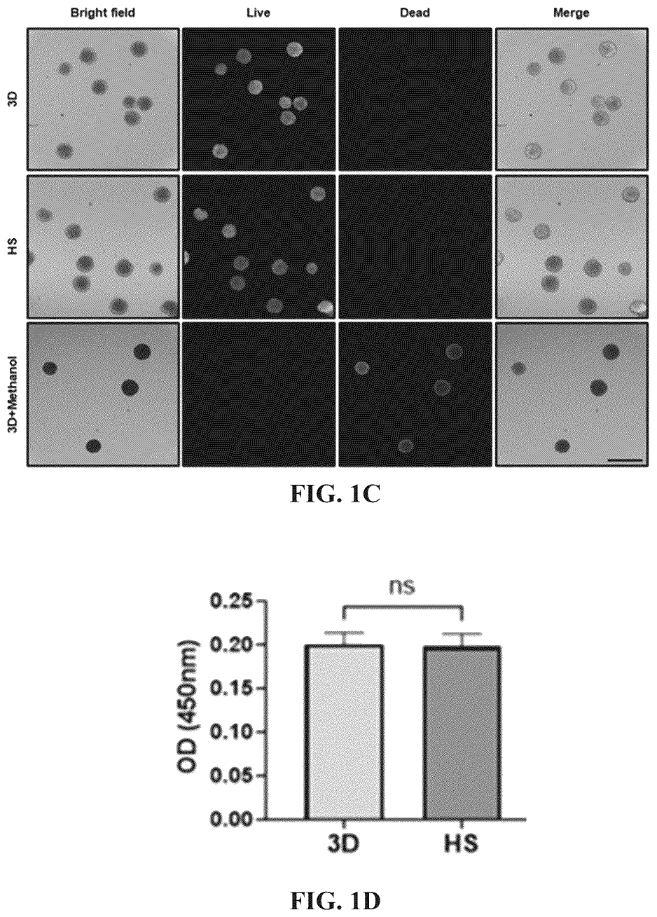

In Example of the present disclosure, the ‘stem cell-drug carrier’ was prepared as shown in FIG. 1A. Specifically, 90 mg of PLGA and 10 mg of melatonin were added to 1 mL of dichloromethane to prepare a first solution, and the first solution was dropped into 5 mL of a 1% PVA (polyvinyl alcohol, Sigma) solution. Thereafter, the mixture was subjected to emulsification and evaporation processes to prepare a ‘melatonin-containing drug carrier.’ The prepared melatonin-containing drug carrier and 1.2×10⁶ mesenchymal stem cells were cultured for 24 hours to produce a ‘stem cell-drug carrier.’

According to another aspect of the present disclosure, the present disclosure provides a composition for regenerating intestinal epithelial cells or a composition for inducing revival stem cells, including the cell-drug carrier.

In a specific embodiment of the present disclosure, it was confirmed that the stem cell-drug carrier induced organoids derived from intestinal epithelium or colon; and revival stem cells in an intestinal inflammation animal model. In addition, it was confirmed that the stem cell-drug carrier of the present disclosure suppressed shortening of colon length and improved an intestinal epithelial structure in the intestinal inflammation animal model. Therefore, the stem cell-drug carrier of the present disclosure may be used in various fields of treatment of intestinal damage disease and inflammatory bowel disease.

The above revival stem cells (revSC) refer to stem cells that induce regeneration of the intestinal epithelium in place of existing Lgr5-expressing stem cells. The revival stem cells may form colonies capable of self-renewal, thereby newly creating a damaged epithelial layer and improving an intestinal epithelial structure.

The reagent composition of the present disclosure may further include at least one known ingredient having an intestinal epithelial regeneration; or revival stem cell induction effect.

According to another aspect of the present disclosure, the present disclosure provides a pharmaceutical composition for preventing or treating intestinal epithelial damage disease; or a pharmaceutical composition for preventing or treating inflammatory bowel disease, including the stem cell-drug carrier.

In a specific embodiment of the present disclosure, the intestinal epithelial damage disease may be at least one selected from the group consisting of Crohn's disease, ulcerative colitis, ulcerative duodenitis, hemorrhagic rectal ulcer, leaky gut syndrome, gastritis, gastric ulcer, pouchitis, enteritis, and ischemic colitis.

In a specific embodiment of the present disclosure, the inflammatory bowel disease may be any one selected from the group consisting of ulcerative colitis, Crohn's disease, collagenous colitis, lymphocytic colitis, ischemic colitis, diversion colitis, and Behcet's syndrome.

The pharmaceutical composition of the present disclosure may be a cell therapeutic agent composition.

In the present disclosure, the cell therapeutic agent refers to a medicine (US FDA regulations) used for treatment, diagnosis, and prevention with cells and tissues prepared through isolation, culture, and special manipulation from a subject, and means a medicine used for treatment, diagnosis and prevention through a series of actions, such as ex vivo proliferating and selecting living autologous, allogeneic, or xenogenic cells to restore the function of cells or tissues, changing the biological characteristics of cells by other methods, and the like.

In addition, the composition of the present disclosure may be prepared using pharmaceutically suitable and physiologically acceptable adjuvants in addition to the active ingredients. As the adjuvants, excipients, disintegrants, sweeteners, binders, coating agents, expanding agents, lubricants, slip modifiers, flavoring agents, or the like may be used.

The composition according to the present disclosure may be formulated as a pharmaceutical composition by further including at least one pharmaceutically acceptable carrier, in addition to the above-described active ingredients for administration. The pharmaceutically acceptable carrier may be used by mixing saline, sterile water, Ringer's solution, buffered saline, a dextrose solution, a maltodextrin solution, glycerol, ethanol, liposome and at least one of these ingredients, and if necessary, other conventional additives such as antioxidants, buffers, bacteriostats, etc. may be added. In addition, diluents, dispersants, surfactants, binders and lubricants may be additionally added to formulate the composition into injectable formulations such as aqueous solutions, suspensions, emulsions, etc., pills, capsules, granules or tablets, and antibodies or other ligands specific to a target organ may be used in combination with the carrier so as to specifically act on the target organ. Furthermore, the composition may be preferably formulated according to each disease or ingredients using an appropriate method in the art or methods disclosed in Remington's literature.

The pharmaceutical composition of the present disclosure may be formulated in the form of a solution, a suspension, a dispersion, an emulsion, a gel agent, an injectable solution, and a sustained-release preparation of an active compound, and preferably an injection.

When the pharmaceutical composition of the present disclosure is formulated into the injection, the pharmaceutical composition may be formulated in a very physically and chemically stable injection by adjusting pH using an acid aqueous solution or a buffer solution such as a phosphate, etc., that can be used as an injection to ensure product stability according to distribution of the injection prescription.

More specifically, the injection may be prepared by dissolving the pharmaceutical composition in water for injection together with a stabilizer or a solubilizing agent, and then sterilizing the pharmaceutical composition, particularly by high-temperature and reduced-pressure sterilization or aseptic filtration. The water for injection may be distilled water for injection or a buffer solution for injection, for example, a phosphate buffer solution or a sodium dihydrogen phosphate (NaH₂PO₄)-citric acid buffer solution in the range of pH 3.5 to 7.5. The phosphate used may be in the form of sodium or potassium salt, or an anhydrous or hydrated product, and may also be in the form of citric acid, or an anhydrous or hydrated product.

In addition, the stabilizer used in the present disclosure includes sodium pyrosulfite, sodium bisulfite, sodium metabisulfite or ethylenediamine tetraacetic acid, and the solubilizing agent includes base such as sodium hydroxide, sodium bicarbonate, sodium carbonate or potassium hydroxide, or acid such as hydrochloric acid or acetic acid.

The injection according to the present disclosure may be formulated to be bioabsorbable, biodegradable, and biocompatible. The bioabsorbability means that the injection may disappear in the body upon initial application, with or without degradation of the dispersed injection. The biodegradability means that the injection may be broken down or degraded in the body by hydrolysis or enzymatic degradation. The biocompatibility means that all of the ingredients are non-toxic in the body.

The injection according to the present disclosure may be prepared using conventional diluents, excipients such as fillers, extenders, binders, wetting agents, and surfactants, or the like.

The composition or active ingredient of the present disclosure may be administered by a conventional method, depending on the purpose, through intravenous, intraarterial, intraperitoneal, intramuscular, intrasternal, transdermal, intranasal, subcutaneous, intrauterine epidural, inhalation, topical, rectal, oral, intraocular, or intradermal routes, and preferably administered intravenously. The composition or active ingredient of the present disclosure may be administered by an injection or catheter.

In the composition of the present disclosure, the dose of the active ingredient may be adjusted in the range of 1×10¹ to 1×10⁵⁰ units/kg, preferably 1×10¹ to 1×10³⁰ units/kg, more preferably 1×10⁵ to 1×10²⁰ units/kg, and most preferably 1×10⁷ to 1×10⁹ units/kg for an adult weighing 60 kg. However, an optimal dose to be administered may be easily determined by those skilled in the art, and may be adjusted according to various factors including a type of disease, the severity of disease, the contents of active ingredients and other ingredients contained in the composition, a type of formulation, age, body weight, general health condition, sex and diet of a patient, an administration time, a route of administration, a secretion rate of a composition, duration of treatment, and drugs to be used concurrently.

According to yet another aspect of the present disclosure, the present disclosure provides a method for preparing a cell therapeutic agent for regenerating intestinal epithelial cells, including (a) preparing a melatonin-containing drug carrier by mixing and homogenizing melatonin and a polymer; and (b) preparing a stem cell-drug carrier by mixing the melatonin-containing drug carrier prepared in step (a) and stem cells.

The cell therapeutic agent prepared by the method of the present disclosure induces regeneration of damaged intestinal epithelial tissue through induction of revival stem cells, and may be used for the prevention or treatment of intestinal damage disease or inflammatory bowel disease.

In a specific embodiment of the present disclosure, the polymer of step (a) may be a biodegradable polymer as described above.

In a specific embodiment of the present disclosure, step (a) may be mixing and homogenizing an aqueous solution; and an oily solution containing melatonin and a polymer.

Duplicated contents are omitted in consideration of the complexity of the present specification, and terms not defined otherwise in the present specification have the meanings commonly used in the art to which the present disclosure pertains.

According to another aspect of the present disclosure, the present disclosure provides a method of treating intestinal epithelial damage disease, comprising: administering the stem cell-drug carrier according to claim 1 to an individual in need thereof. And the present disclosure provides a method of treating inflammatory bowel disease, comprising: administering the stem cell-drug carrier according to claim 1 to an individual in need thereof.

In a specific embodiment of the present disclosure, the subject may be an individual who is expected to develop an intestinal epithelial damage disease or inflammatory bowel disease; who has developed the disease; or who has been diagnosed with the disease.

Hereinafter, the present disclosure will be described in more detail through Examples. These Examples are just illustrative of the present disclosure, and it will be apparent to those skilled in the art that it is not interpreted that the scope of the present disclosure is limited to these Examples.

### Example 1. Preparation of Stem Cell-Drug Carrier

### 1-1. Preparation of Heterospheroids

Heterospheroids (HS) were stem cell-drug carriers including melatonin-containing drug carriers; and stem cells, which were prepared as shown in FIG. 1A. The heterospheroids were cultured in a 3D form of mesenchymal stem cells secreting a large amount of PGE2 and melatonin microspheres, and targeted to use a therapeutic material for binding PGE2 and melatonin.

Specifically, to prepare the heterospheroids (HS), a solution of 90 mg of PLGA (Sigma) and 10 mg of melatonin dissolved in 1 ml of dichloromethane (Junsei Chemical) was dropped to 5 mL of a 1% PVA (polyvinyl alcohol, Sigma) solution. After the dropping, the mixture was emulsified at 13,000 rpm for 5 minutes. The emulsion was then transferred to 60 mL of a 1% PVA aqueous solution and stirred at room temperature for 5 hours to evaporate the organic solvent, thereby obtaining ‘melatonin microspheres.’

Umbilical cord blood-derived mesenchymal stem cells were cultured in a KSB-3 medium (Kangstem Biotech). 1.2×10⁶ cultured umbilical cord blood-derived mesenchymal stem cells and 704 μg of melatonin microspheres were mixed in 2 mL of the KSB-3 medium. The mixture was then cultured in AggreWell™400 Microwell Culture Plates 24-well plate (STEMCELL Technologies) for 24 hours to generate 3D mesenchymal stem cells (3D-MSCs) and heterospheroids (HS). After culturing, the cell culture dish was washed with PBS to remove the mesenchymal melatonin microspheres. The heterospheroids included 1×10³ cells and melatonin microspheres.

As control groups for an experiment to be described below, 2D mesenchymal stem cells (2D) and 3D mesenchymal stem cells (3D) were used.

### 1-2. Characteristic Analysis of Heterospheroids

For characteristic analysis of the prepared 3D-MSC and HS, diameter measurement, LIVE/DEAD staining, and CCK-8 analysis were performed, and the results were shown in FIGS. 1B to 1D, respectively.

As shown in FIG. 1B, it was confirmed that the average diameter of the heterospheroids (HS) was 162.09 μm.

As shown in FIGS. 1C and 1D, it was confirmed that the cell viability of HS was similar to that of the 3D mesenchymal stem cell (3D).

### Example 2. Effect of Heterospheroids on Inducing Revival Stem Cells

### 2-1. Co-Culture of Heterospheroids and Intestinal Epithelial Organoids

The induction of revival stem cells in the intestinal epithelium of the heterospheroids prepared in Example 1 was confirmed. Specifically, the intestinal epithelial organoids were seeded on a Matrigel and then cultured for 24 hours. One heterospheroid was added to each crypt containing the intestinal epithelial organoid and co-cultured for 48 hours. A control group was added with 1×10³ 2D mesenchymal stem cells (2D) or one 3D mesenchymal stem cell (3D) per each crypt.

### 2-2. Morphological Analysis

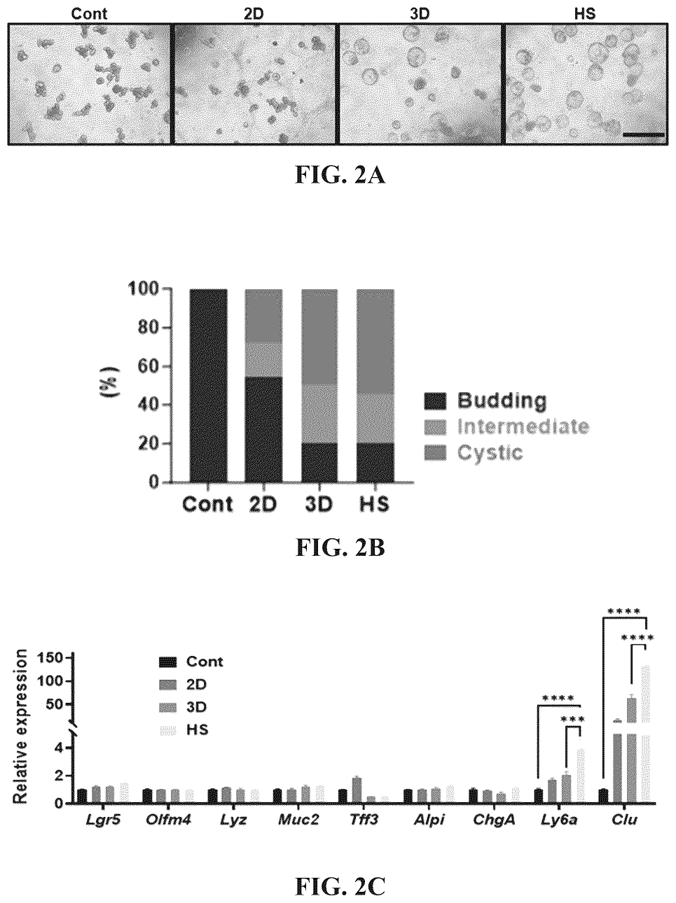

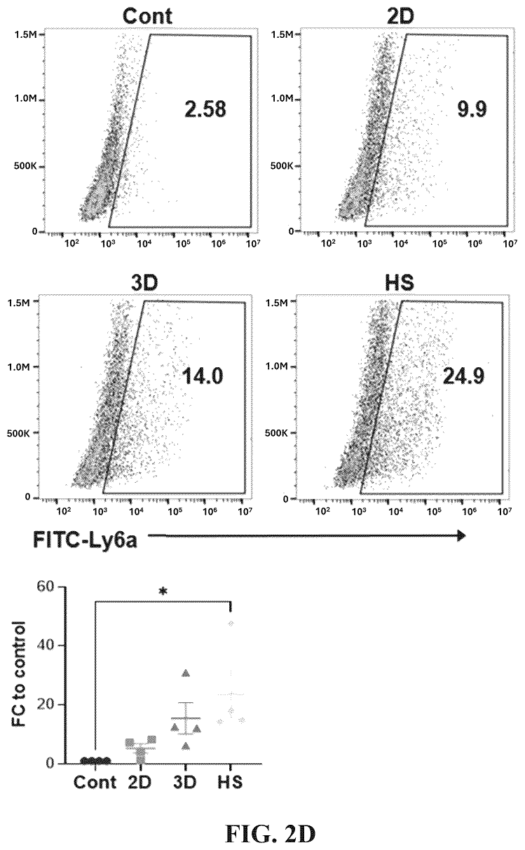

After co-culturing heterospheroids and intestinal epithelial organoids as in Example 2-1 above, the cell morphology was analyzed using a microscope. The microscopic observation results were shown in FIG. 2A, and the results of classifying the cell morphology based on the microscopic observation results were shown in FIG. 2B.

As shown in FIGS. 2A and 2B, as a result of co-culturing the heterospheroids and the intestinal epithelial organoids, it was confirmed that the organoids co-cultured with the heterospheroids had a more prominent change to a cystic form than the control groups 2D and 3D.

### 2-3. Analysis of Expression of Intestinal Epithelial Markers

After co-culturing the heterospheroids and the intestinal epithelial organoids as in Example 2-1 above, the expression of revival stem cell markers Ly6a and Clu, stem cell markers Lgr5 and Olfm4, and intestinal epithelial markers Lyz, Muc2, Tff3, Alpi, and ChgA was analyzed using Real-time qPCR. The results of analyzing the expression of the markers were shown in FIG. 2C.

As shown in FIG. 2C, the expression of revival stem cell markers Ly6a and Clu in a heterospheroid-treated group (HS) and a 3D mesenchymal stem cell-treated group (3D) was significantly higher than that in a negative control group (cont.) and a 2D mesenchymal stem cell-treated group (2D). In particular, the expression of revival stem cell markers Ly6a and Clu in the heterospheroid-treated group was 1.92 times and 2.09 times higher than that in the 3D mesenchymal stem cell-treated group (3D), respectively.

### 2-4. Flow Cytometry

After co-culturing the heterospheroids and the intestinal epithelial organoids as in Example 2-1 above, cells (i.e., revival stem cells) expressing a revival stem cell marker Ly6a were analyzed by flow cytometry. The flow cytometry results were illustrated in FIG. 2D.

As shown in FIG. 2D, it was confirmed that the heterospheroid-treated group (HS) included the most revival stem cells in the intestinal epithelial organoids. In particular, it was confirmed that the heterospheroid-treated group (HS) included about 1.8 times more revival stem cells than the 3D mesenchymal stem cell-treated group (3D) to induce more revival stem cells than 3D mesenchymal stem cells.

### Example 3. Confirmation of Protective Effect By Heterospheroid Administration in Intestinal Inflammation Mouse Model

### 3-1. Preparation of Intestinal Inflammation Mouse Model and Heterospheroids

To prepare an intestinal inflammation mouse model, 9-week-old C57BL/6 mice were supplied with drinking water containing 2.5% DSS for 5 days to induce intestinal inflammation.

In addition, heterospheroids were prepared using mesenchymal stem cells derived from umbilical cord blood (UCB) and tonsil (T) as in Example 1-1. The prepared heterospheroids were named HS#UCB and HS#T, respectively.

In order to evaluate the in vivo efficacy of the prepared heterospheroids, 2,000 heterospheroids (1,000 cells per spheroid) were injected intraperitoneally per mouse on day 5 of supplying drinking water containing DSS. In order to evaluate the regenerative effect on the intestinal epithelium that was already damaged by the induced inflammation, the time point of cell administration was set to day 5, not the initial induction.

In a positive control group, 3D mesenchymal stem cells (3D) derived from umbilical cord blood (UCB) and tonsil (T) were injected into the intestinal inflammation mouse model.

### 3-2. Analysis of Body Weight and Survival Rate of Intestinal Inflammation Mouse Model

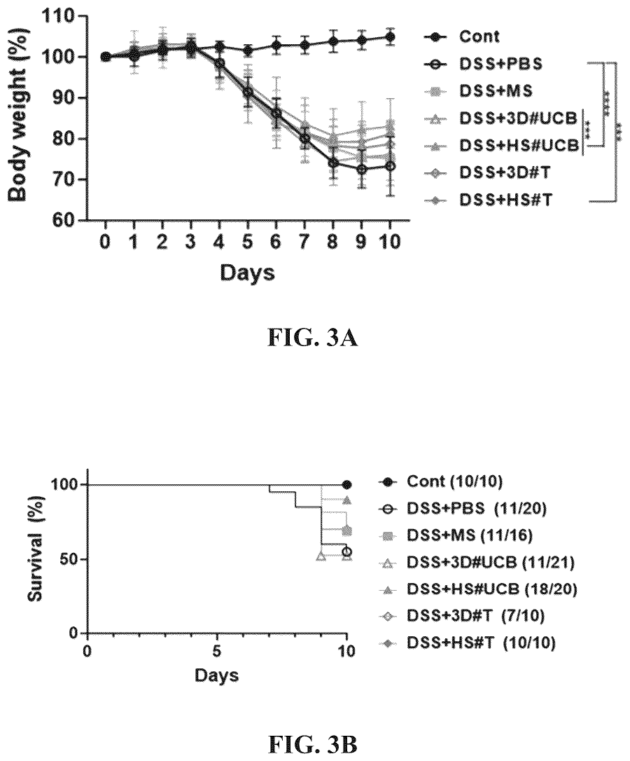

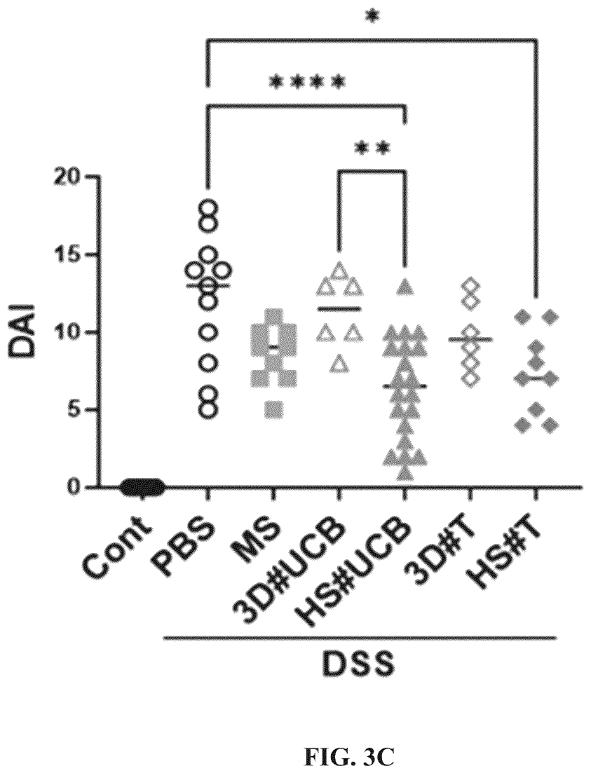

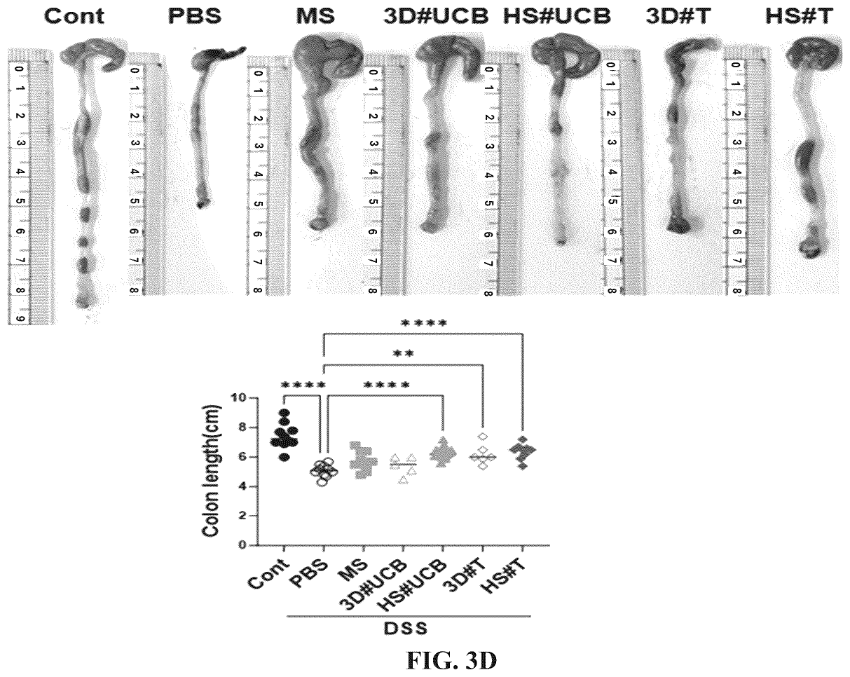

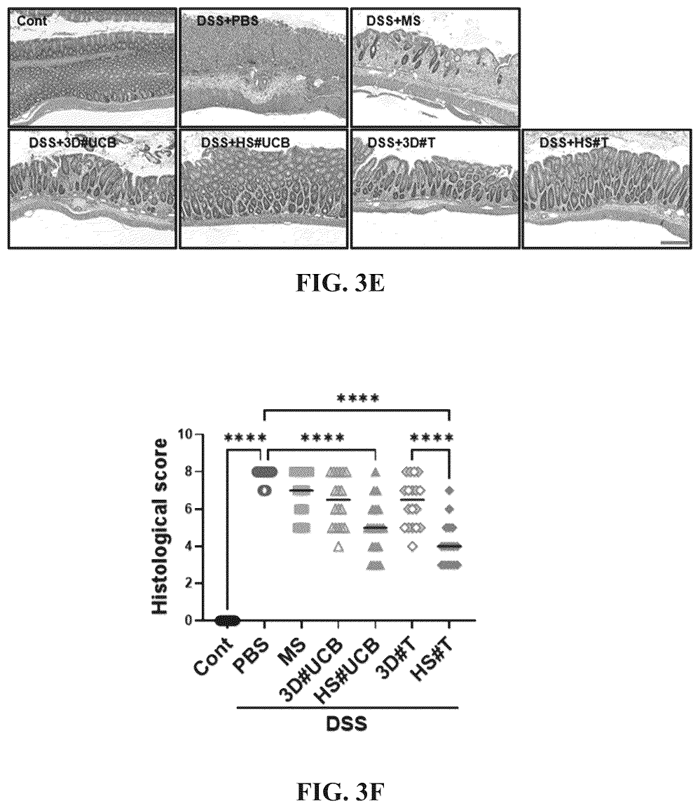

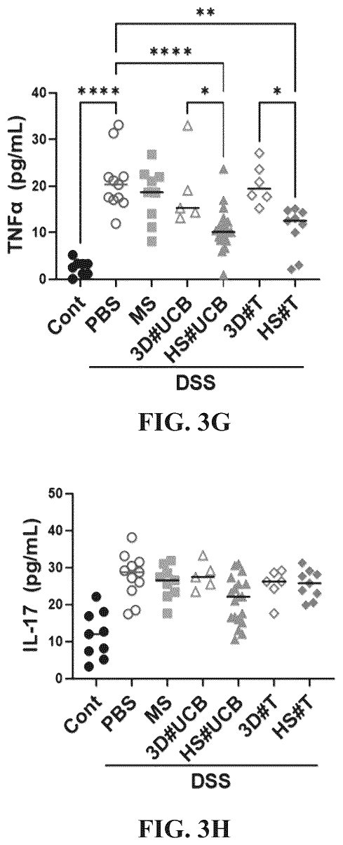

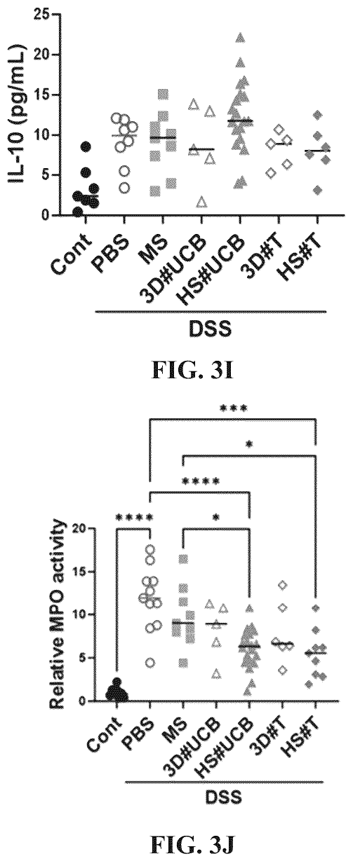

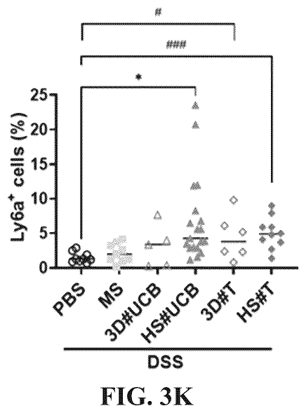

As in Example 3-1 above, the body weight and survival rate of the intestinal inflammation mouse model administered with heterospheroids were confirmed. The results of measuring body weights were shown in FIG. 3A, and the results of confirming the survival rates were shown in FIG. 3B.

As shown in FIGS. 3A and 3B, a HS#UCB-administered group and a HS#T-administrated group showed less weight loss than a PBS-administered group (DSS+PBS). In particular, the HS#UCB-administered group showed a significantly low weight loss rate and the highest survival rate.

### 3-3. Evaluation of Colitis Severity in Intestinal Inflammation Mouse Model

The severity of colitis in an intestinal inflammation mouse model was evaluated on day 10 of DSS administration through evaluation of disease activity index (DAI). The results of evaluating the disease activity index of colitis were shown in FIG. 3C.

As shown in FIG. 3C, the disease activity indexes in the HS#UCB and HS#T-administered groups were statistically significantly lower than that of the PBS control group, and in particular, HS#UCB showed a significantly lower disease activity index than a 3D#UCB-administered group. The results meant that the severity of colitis was alleviated when HS#UCB and HS#T were administered.

The intestinal inflammation therapeutic effect according to HS#UCB and HS#T administration was evaluated by measuring the colon length in an intestinal inflammation mouse model and improving intestinal epithelial damage through H&E staining. The results of measuring the colon length were shown in FIG. 3D, and the results of evaluating intestinal epithelial damage were shown in FIGS. 3E and 3F.

As shown in FIG. 3D, it was confirmed that the HS#UCB-administered group and the HS#T-administered group protected the colon length from being shortened (i.e., alleviation of colitis symptoms).

As shown in FIGS. 3E and 3F, the HS#UCB-administered group and the HS#T-administered group showed improvement in intestinal epithelial structure and inflammatory cell infiltration.

### 3-5. Analysis of Inflammatory Indicators in Intestinal Inflammation Mouse Model

As in Example 3-1 above, inflammatory indicators TNF-α, IL-17, and IL-10 in the intestinal inflammation mouse model administered with heterospheroids were analyzed, and the results were shown in FIGS. 3G to 3I.

As shown in FIGS. 3G to 3I, the HS#UCB and HS#T-administered groups showed significant decreases in TNF-α and IL-17 levels, and increases in IL-10 level. This means that the HS#UCB and HS#T administration regulates the inflammatory response of the intestinal inflammation mouse model.

### 3-6. Analysis of Myeloperoxidase (MPO) Activity in Colon Tissue of Intestinal Inflammation Mouse Model

MPO is an enzyme found in neutrophil granules and is used as an indicator of neutrophil infiltration and inflammatory activity. In colitis, MPO activity is highly correlated with intestinal damage levels. As in Example 3-1 above, the MPO activity was analyzed in the colon of an intestinal inflammation mouse model administered with heterospheroids, and the results were shown in FIG. 3J.

As shown in FIG. 3J, it was confirmed that the HS#UCB and HS#T-administered groups showed a significant decrease in MPO activity compared to the PBS group. This means that HS reduces inflammation and intestinal damage.

### 3-7. Evaluation of Induction of Revival Stem Cells in Intestinal Inflammation Mouse Model

Ly6a-expressing cells were analyzed in an intestinal inflammation mouse model administered with heterospheroids as in Example 3-1 above by flow cytometry. The flow cytometry results were illustrated in FIG. 3K.

As shown in FIG. 3K, it was confirmed that the HS#UCB and HS#T-administered groups showed a significant increase in Ly6a-expressing cells compared to the PBS group. The results mean that HS contributes to intestinal epithelial regeneration by inducing revival stem cells in intestinal epithelium already damaged by inflammation.

### Example 4. Comparison Experiment on Revival Stem Cell Induction Ability of Heterospheroids According To Drug Type

### 4-1. Preparation of Heterospheroids According To Drug Type

In the art, quercetin is known as a therapeutic agent for inflammatory bowel disease. Accordingly, in Example 4, the revival stem cell induction ability of heterospheroids (hereinafter referred to as ‘Mel-HS’) including melatonin microspheres and 3D mesenchymal stem cells prepared in Example 1-1; and heterospheroids (hereinafter referred to as ‘Q-HS’) including quercetin microspheres and 3D mesenchymal stem cells was compared. The Q-HS was prepared by the same method as Example 1-1, but using quercetin instead of melatonin.

### 4-2. Comparison of Revival Stem Cell Induction Ability

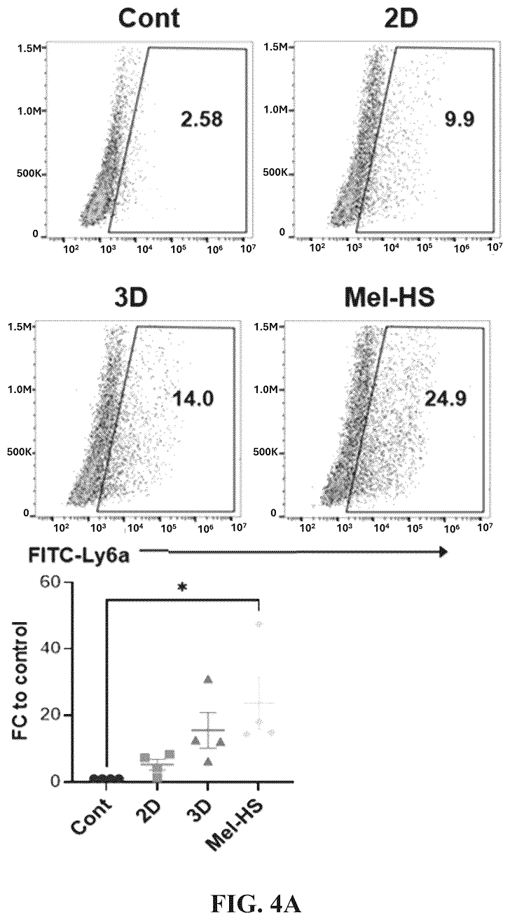

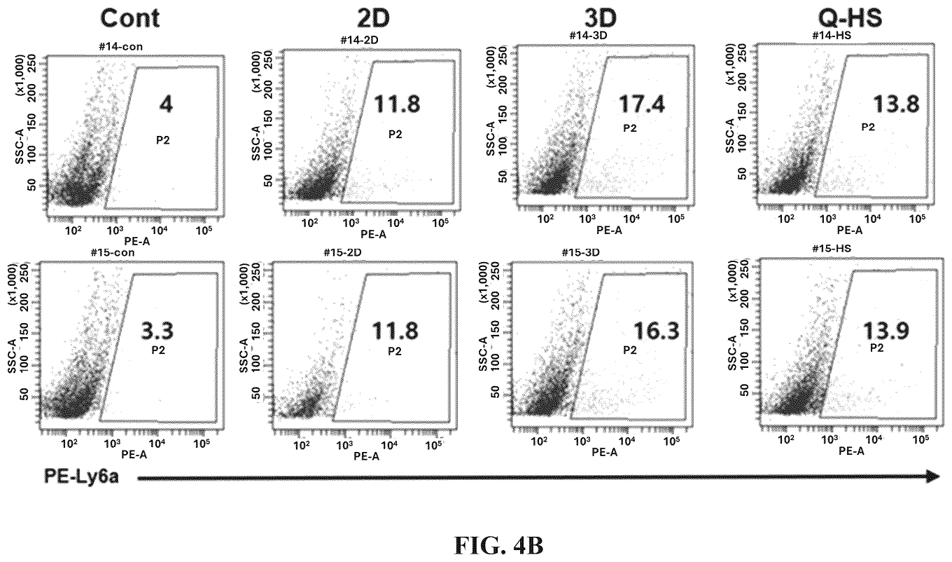

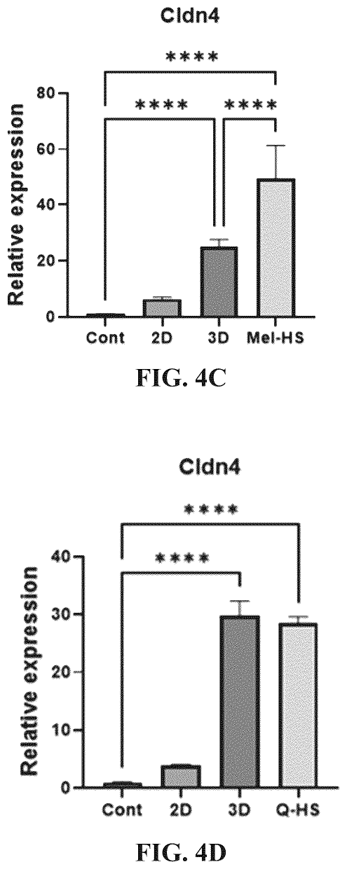

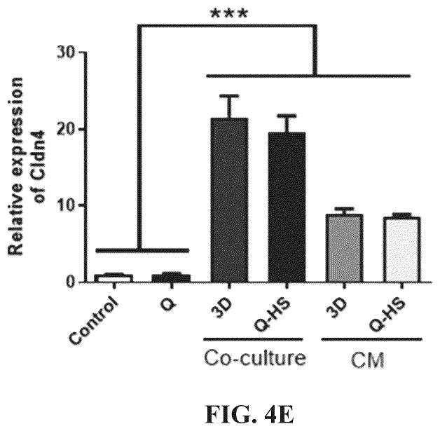

Mel-HS was treated in small intestine epithelial organoids and then co-cultured for 48 hours. After culturing, cells expressing a revival stem cell marker Ly6a of the small intestine epithelial organoids were analyzed by flow cytometry. In addition, a Q-HS-treated group was also performed in the same manner as described above. As a control group for the experiment, 2D mesenchymal stem cells (2D) or 3D mesenchymal stem cells (3D) were treated. The flow cytometry results of the Mel-HS-treated group were shown in FIG. 4A, and the flow cytometry results of the Q-HS-treated group were shown in FIG. 4B.

As shown in FIGS. 4A and B, it was confirmed that in the Mel-HS-treated group, Ly6a expressing cells in organoids were significantly increased than the control groups 2D and 3D (FIG. 4A). However, it was confirmed that in the Q-HS-treated group, the Ly6a expressing cells in organoids were significantly reduced than the control groups 2D and 3D (FIG. 4B). The result means that quercetin does not affect revival stem cell induction, and the revival stem cell induction effect by Mel-HS is a specific effect.

### 4-3. Analysis of Expression of Revival Stem Cell Markers In Organoids

Mel-HS was treated in small intestine epithelial organoids and then co-cultured for 48 hours. After culturing, the expression of a revival stem cell marker Claudin-4 (Cldn4) was measured through real-time qPCR. In addition, a Q-HS-treated group was also performed in the same manner as described above. As a control group for the experiment, 2D mesenchymal stem cells (2D) or 3D mesenchymal stem cells (3D) were treated. The real-time qPCR result of the Mel-HS-treated group was shown in FIG. 4C, and the real-time qPCR result of the Q-HS-treated group was shown in FIG. 4D.

As shown in FIGS. 4C and 4D, it was confirmed that in the Mel-HS-treated group, the expression of Cldn4 was significantly increased than the control groups 2D and 3D (FIG. 4C).

However, it was confirmed that in the Q-HS treated group, the expression of Cldn4 was higher than in the control group 2D, but lower than in the control group 3D. This result indicates that quercetin does not promote a revival stem cell induction effect of stem cells.

In addition, Q-HS was administered and co-cultured to mouse colon organoids in the same manner as described above, and the expression of Cldn4 was analyzed through real-time qPCR.

A control group CM (conditioned media) of the experiment is a group treated with a stem cell culture. Real-time qPCR results were shown in FIG. 4E.

As shown in FIG. 4E, it was confirmed that in Q-HS, the expression of Cldn4 was lower than in the control group 3D even in mouse colon organoids.

The result means that the melatonin of Mel-HS specifically increases the revival stem cell induction efficiency with 3D mesenchymal stem cells. On the other hand, it is meant that, unlike Mel-HS, Q-HS does not show a synergic effect with the 3D mesenchymal stem cells.

Overall, the present inventors prepared the ‘stem cells-drug carriers (i.e., heterospheroids)’ by mixing melatonin microspheres and 3D mesenchymal stem cells. It was confirmed that the heterospheroids of the present disclosure continuously released PGE2 and had excellent revival stem cell induction ability. This means that the heterospheroids of the present disclosure have an excellent intestinal epithelium regeneration effect, and the heterospheroids of the present disclosure may be used in various fields of treatment of intestinal damage disease and inflammatory bowel disease.

As described above, specific parts of the present disclosure have been described in detail, and it will be apparent to those skilled in the art that these specific techniques are merely preferred embodiments, and the scope of the present disclosure is not limited thereto. Therefore, the substantial scope of the present disclosure will be defined by the appended claims and their equivalents.

1. A stem cell-drug carrier comprising a melatonin-containing drug carrier; and a stem cell.

2. The stem cell-drug carrier of claim 1, wherein the melatonin-containing drug carrier is prepared with one or more polymers selected from the group consisting of polylactide-co-glycolide, polylactide-co-glycolide-co-ethylene glycol, polystyrene-co-ethylene glycol, polyethyleneimine-co-ethylene glycol, polyphosphagen-co-ethylene glycol, polylactide-co-ethylene glycol, polycaprolactone-co-ethylene glycol, polyanhydride-co-ethylene glycol, polymalic acid-co-ethylene glycol and derivatives thereof, polyalkylcyanoacrylate-co-ethylene glycol, polyhydroxybutyrate-co-ethylene glycol, polycarbonate-co-ethylene glycol and polyorthoester-co-ethylene glycol, polyethylene glycol, poly-L-lysine-co-ethylene glycol, polyglycolide-co-ethylene glycol, polymethylmethacrylate-co-ethylene glycol, polyvinylpyrrolidone-co-ethylene glycol, and copolymers thereof.

3. The stem cell-drug carrier of claim 1, wherein the stem cell is an embryonic stem cell, a mesenchymal stem cell or an induced pluripotent stem cell.

4. The stem cell-drug carrier of claim 3, wherein the mesenchymal stem cell is derived from embryonic yolk sac, placenta, umbilical cord, umbilical cord blood, tonsil, skin, peripheral blood, bone marrow, adipose tissue, muscle, liver, nerve tissue, periosteum, fetal membrane, synovium, synovial fluid, amniotic membrane, meniscus, anterior cruciate ligament, articular chondrocytes, milk teeth, perivascular cells, trabecular bone, subpatellar fat pad, spleen or thymus.

5. The stem cell-drug carrier of claim 1, wherein the stem cell is in a form of a spheroid.

6. A method of regenerating intestinal epithelial cells, comprising:
administering the stem cell-drug carrier according to claim 1 to an individual in need thereof.

7. The method of claim 6, wherein the regenerating intestinal epithelial cell is treating intestinal epithelial damage disease.

8. The method of claim 7, wherein the intestinal epithelial damage disease is at least one selected from the group consisting of Crohn's disease, ulcerative colitis, ulcerative duodenitis, hemorrhagic rectal ulcer, leaky gut syndrome, gastritis, gastric ulcer, pouchitis, enteritis, and ischemic colitis.

9. A method of inducing revival stem cells comprising:
administering the stem cell-drug carrier according to claim 1 to an individual in need thereof.

10. The method of claim 9, wherein the revival stem cell is a leucine-rich repeat-containing G-protein coupled receptor 5 (Lgr5)-positive epithelial stem cell.

11. A method of treating inflammatory bowel disease, comprising:
administering the stem cell-drug carrier according to claim 1 to an individual in need thereof.

12. The method of claim 11, wherein the inflammatory bowel disease is any one selected from the group consisting of ulcerative colitis, Crohn's disease, collagenous colitis, lymphocytic colitis, ischemic colitis, diversion colitis, and Behcet's syndrome.

13. A method for preparing a cell therapeutic agent for regenerating intestinal epithelial cells, comprising:
(a) preparing a melatonin-containing drug carrier by mixing and homogenizing melatonin and a polymer; and
(b) preparing a stem cell-drug carrier by mixing the melatonin-containing drug carrier prepared in step (a) and stem cells.
